(19) United States
(12) Patent Application Publication
(10) Pub. No.: US 2011/0038919 A1
(43) Pub. Date: Feb. 17, 2011

(54) TEXTILE MATERIALS WITH ADHESIVE AND METHODS FOR USE THEREOF

(75) Inventors: Kevin C. Tauer, North Mankato, MN (US); Charles E. Boyer, Elysian, MN (US)

Correspondence Address: COLOPLAST A/S;Attention: Corporate Patents, Holtedam 1, DK-3050 Humlebaek, DK

(73) Assignee: COLOPLAST A/S, Humlebaek (DK)

(21) Appl. No.: 12/865,396

(22) PCT Filed: Jan. 27, 2009
(86) PCT No.: PCT/DK09/50026

§ 371 Date: Oct. 22, 2010

Related U.S. Application Data
(60) Provisional application No. 61/025,105, filed on Jan. 31, 2008.

(51) Int. Cl. A61K 9/70; A61K 31/74; A61K 33/38; A61P 31/00

(52) U.S. Cl. 424/443; 424/78.07; 424/618

(57) ABSTRACT

Textile materials for management of skin health complications associated with skin folds, as well as adhesives for attaching the materials to the skin of a subject. The adhesive can be supplied separately from the material, for application post-production, or can be applied to the material during production.

### TECHNICAL FIELD

This document relates generally to textile materials and adhesives, and in particular to textile materials and adhesives for management of skin health.

### BACKGROUND

Skin folds have been problematic for health care providers caring for bariatric patients, among others. Skin folds can occur, for example, when an individual is overweight, with “baby fat” in children, or under sagging breasts. A complication of skin folds is that moisture tends to build-up between skin layers, which can lead to infection and odor, as well as other problems such as, for example, inverse or flexural psoriasis (see, e.g., U.S. Publication No. 2003/0185915), intertrigo (a superficial dermatitis caused by moisture, friction, warmth and sweat retention and characterized by erythema, maceration, burning, itching and sometimes erosions, fissures and exudations, and secondary infections), candidiasis, erythrasma, folliculitus, and tinea cruris (a fungal infection of the perineum also known as “jock itch”).

### SUMMARY

This document provides materials and methods for managing dermatological complications that can be associated with skin folds. The materials and methods can promote translocation of moisture, and thus can be used to manage skin health complications associated with skin folds, including candidiasis, erythrasma, folliculitus, tinea cruris, intertrigo, odor, inflammation, and infection by bacteria and/or fungi. As used herein, the term “translocation” includes any means by which moisture is not only wicked or transported away from a skin fold, but also undergoes a phase change from a liquid to a gas or otherwise evaporates. The materials provided herein are effective for translocation of moisture from skin folds, and thus are effective to protect skin and promote healing in the area of skin folds. The materials also are easy to use, relatively inexpensive, and may be disposable.

In one aspect, this document features a textile material having top and bottom surfaces and comprising a polyurethane coating, wherein the textile material is adapted for placement within a skin fold for management of one or more skin health complications associated therewith, and wherein the textile material has an adhesive on one or both of the top and bottom surfaces. The textile material can have a width from about 1 inch to about 36 inches (or, about 2.54 cm to about 91.44 cm) and a length of about 3 inches to about 1,000 feet (or, about 7.62 cm to about 304.8 m). The textile material can further comprise an antimicrobial silver complex (e.g., a silver ion-exchange resin, a silver zeolite, a silver exchanged on calcium phosphate, a silver glass). The antimicrobial silver complex can be silver sodium hydrogen zirconium phosphate. The antimicrobial silver complex can be present at a concentration of from 0.01 to 0.10 mg/cm2. The one or more skin health complications can include moisture, candidiasis, erythrasma, folliculitus, tinea cruris, intertrigo, odor, inflammation, bacteria, fungi, or any combination thereof. The adhesive can be pattern coated onto only one of the top and bottom surfaces of the textile material.

In another aspect, this document features a method for using a textile material for management of one or more skin health complications associated with a skin fold, comprising the steps of: placing a piece of adhesive onto a textile material dimensioned such that at least one portion of the textile material is exposed to air outside of the skin fold; and placing the textile material over skin of the skin fold, such that the adhesive is in contact with the skin, and such that the skin fold envelopes the textile material with the at least one portion thereof being exposed to air. The textile material can comprise a polyurethane coating and/or an antimicrobial silver complex (e.g., a silver ion-exchange resin, a silver zeolite, a silver exchanged on calcium phosphate, or a silver glass). The antimicrobial silver complex can be silver sodium hydrogen zirconium phosphate. The method can further comprise performing a routine skin inspection, using a skin cleanser (e.g., a skin cleanser having properties compatible with the translocation function of the textile material) to topically clean the skin fold, using an antifungal powder to topically cover the skin fold, determining approximate dimensions of the skin fold, and/or separating the skin fold after a selected time has elapsed, to assess skin health in proximity to the skin fold.

In another aspect, this document features an article of manufacture comprising: a textile material having top and bottom surfaces and comprising a polyurethane coating, wherein the textile material is adapted for placement within a skin fold for management of one or more skin health complications associated therewith; and an adhesive. The textile material can comprise an antimicrobial silver complex (e.g., a silver ion-exchange resin, a silver zeolite, a silver exchanged on calcium phosphate, or a silver glass). The antimicrobial silver complex can be silver sodium hydrogen zirconium phosphate. The adhesive can be a transfer adhesive or a double-sided tape. The adhesive can be in a roll or in a pad. The article of manufacture can comprise a plurality of pieces of adhesive of varying size.

In yet another aspect, this document features an article of manufacture comprising: a textile material having top and bottom surfaces, the textile material comprising a polyurethane coating and an antimicrobial silver complex, wherein the textile material is adapted for placement within a skin fold for management of one or more skin health complications associated therewith, and wherein the antimicrobial silver complex is silver sodium hydrogen zirconium phosphate; and a roll of transfer adhesive or double-sided tape.

This document also features an article of manufacture comprising a textile material, the textile material having top and bottom surfaces and comprising a polyurethane coating, wherein the textile material is adapted for placement within a skin fold for management of one or more skin health complications associated therewith, and wherein the textile material has an adhesive pattern coated onto one or both of the top and bottom surfaces. The textile material can comprise an antimicrobial silver complex (e.g., a silver ion-exchange resin, a silver zeolite, a silver exchanged on calcium phosphate, or a silver glass). The antimicrobial silver complex can be silver sodium hydrogen zirconium phosphate.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIGS. 1A and 1B are drawings of a transfer adhesive in an unrolled configuration (FIG. 1A) and a rolled configuration (FIG. 1B).

FIG. 2 is a drawing of a release liner sheet having a plurality of adhesive tape patches attached thereto.

FIG. 3 is a drawing of a textile material having patches of adhesive tape attached thereto.

FIGS. 4A and 4B are drawings of a textile material having adhesive applied thereto in a stripe pattern (FIG. 4A) or a spotted pattern (FIG. 4B). FIG. 4C is a drawing of a textile material having a release liner attached thereto.

### DETAILED DESCRIPTION

The methods and materials provided herein can be effective for management of moisture, candidiasis, erythrasma, folliculitus, tinea cruris, intertrigo, odor, inflammation, bacteria, fungi, or any other complications that may be associated with skin folds. In some embodiments, a textile material for management of skin health complications associated with skin folds can be an INTERDRY Ag brand textile (Coloplast Corp., Minneapolis, Minn.). The material can comprise any suitable polymer, including, for example, polyester. Such a material can be disposable, and can have any suitable dimensions. For example, the dimensions of the textile material can range from 1 inch to 36 inches (or, 2.54 cm to 91.44 cm) in width (e.g., 1, 2, 3, 4, 5, 6, 8, 10, 12, 14, 15, 16, 18, 20, 22, 24, 25, 26, 28, 30, 32, 34, or 36 inches in width) (or, e.g., 2.54, 5.08, 7.62, 10.16, 12.70, 15.24, 20.32, 25.40, 30.48, 35.56, 38.10, 40.64, 45.72, 50.80, 55.88, 60.96, 63.50, 66.04, 71.12, 76.20, 81.28, 86.36, or 91.44 cm in width), and from 3 inches to 1000 feet (or, 7.62 cm to 304.8 m) in length (e.g., 3, 6, 8, 9, 12, 15, 18, 24, or 36 inches, or 4, 6, 8, 10, 12, 16, 20, 50, 100, 200, 250, 300, 400, 500, 600, 700, 750, 800, 900, or 1000 feet in length) (or, e.g., 7.62, 15.24, 20.32, 22.86, 30.48, 38.10, 45.72, 60.96, or 91.44 cm, or 1.22, 1.83, 2.44, 3.05, 3.66, 4.88, 6.10, 15.24, 30.48, 60.96, 76.20, 91.44, 121.92, 152.4, 182.88, 213.36, 228.6, 243.84, 274.32, or 304.80 m in length).

In some embodiments, a textile material can have a polyurethane coating that can, for example, facilitate translocation of moisture. In some embodiments, the material additionally or alternatively can comprise (e.g., can be impregnated or coated with) an antimicrobial silver-containing compound. Suitable silver-containing compounds include, for example, compounds that are ion-exchange resins, zeolites, or substituted glass compounds that release the particular metal ion bonded thereto in the presence of other anionic species. In some embodiments, the silver-containing compound can be an antimicrobial silver sodium hydrogen zirconium phosphate (available from Milliken & Company, Spartanburg, S.C., under the trade name ALPHASAN®). Other useful silver-containing antimicrobials include silver zeolites such as those available from Sinanen (Tokyo) under the trade name ZEOMIC® AJ, silver exchanged on calcium phosphate available from Sangi (Tokyo) under the trade name APISCIDER®, and silver glass, such as those available from Ishizuka Glass Company, Ltd. (Iwakura, Japan) under the trade name IONOPURE®. Such compounds can be used alone or in combination with other silver-containing compounds. For example, combinations of such silver-containing materials can be used to adjust the silver release rate over time. In some embodiments, the materials provided herein can include silver sodium hydrogen zirconium phosphate at a concentration ranging from about 0.01 mg/cm2 to about 0.10 mg/cm² (e.g., 0.01, 0.02, 0.025. 0.03, 0.04, 0.05, 0.06, 0.07. 0.075, 0.08, 0.09, or 0.10 mg/cm²).

In some embodiments, a textile material as provided herein additionally can have a silver vapor coating, which can be provided by electronic, chemical, or other means. Such a coating can be, for example, the ACTICOAT® brand antimicrobial metal coating of Smith & Nephew, Inc. (London), the X-STATIC® brand silver fiber of Noble Biomaterials, Inc. (Scranton, Pa.), or the SILVERLON® brand silver-plated fabrics of Argentum Medical, LLC (Willowbrook, Ill.).

Methods for using the materials provided herein can include any of the following steps: performing inspection of a skin fold and the adjacent areas of skin; using a skin cleanser to topically clean the skin fold; using an antifungal powder to topically cover the skin fold; determining the approximate dimensions of the skin fold; providing a textile material in dimensions such that at least one selected portion of the textile material can be exposed to air outside of the skin fold (e.g., by measuring and cutting a length of the textile material that allows for at least about 1 inch (e.g., about 1 inch, about 1.5 inches, about 2 inches, about 3 inches, or more than 3 inches) (or, e.g., about 2.54 cm, about 3.81 cm, about 5.08 cm, about 7.62 cm, or more than 7.62 cm) of the material to be exposed to air outside the skin fold for moisture evaporation); and placing the textile material within the skin fold, such that the skin fold envelopes the textile material with the at least one selected portion being exposed to air. In addition, the skin fold can be separated after a selected time has elapsed, to assess skin health in proximity to the skin fold. The skin cleanser can be any suitable cleanser having properties compatible with a translocation function of the textile (e.g., normal saline or the SEA-CLENS® brand cleanser from Coloplast Corp.). The antifungal powder can be, for example, any suitable over-the-counter antifungal preparation (e.g., the MICRO-GUARD® brand antifungal powder from Coloplast Corp.). The textile material may be left in place for any length of time (e.g., 1 hour, 2 hours, 4 hours, 6 hours, 8 hours, 12 hours, 24 hours, 36 hours, 2 days, 3 days, 4 days, 5 days, or more than 5 days), depending on the amount of moisture, the general skin condition, and whether a wound dressing also is being used at the site.

It is to be appreciated that the textile material could be sewn or otherwise fashioned into a sock or wrap for covering a body appendage where skin health complications, such as those that can be associated with a skin fold, occur. Further, any suitable method of attachment or closure can be used with the materials provided herein. Attachment means can include, without limitation, tapes (e.g., paper, silk, or medical tape), ostomy pouch fasteners, VELCRO® brand (or equivalent) fasteners, and/or clips (e.g., plastic or metal clips, such as those used for securing elastic bandages and wraps). Other suitable attachment means include adhesives that can be applied to the material during production or post-production, or otherwise.

Any suitable type of adhesive material can be used. Suitable adhesives typically are skin-friendly and atraumatic, such that they can be removed from the skin without causing trauma to the skin or pain to the subject. Useful adhesives include, without limitation, silicone adhesives, acrylic adhesives, hydrogel adhesives, and hydrocolloid adhesives. Adhesive materials can comprise caoutchouc, caoutchouc-like synthetic homo-, co-, or block-copolymers, polyacrylate and copolymerisates thereof, polyurethane, silicone, polyphenylene oxide, acrylate blockcopolymer polyisobutylene, polyvinyl ether, and natural or synthetic resins or mixtures thereof, optionally containing zinc oxide. In some embodiments, adhesives can be medical grade barrier adhesives such as the formulations disclosed in, for example, U.S. Pat. Nos. 4,367,732, 5,051,259, 5,714,225, 6,171,594, 6,303,700, 6,451,883, or 6,437,038, or in published PCT Application No. WO 00/54820 or WO 01/05340, or pressure sensitive adhesive compositions such as those described in WO 89/05619 or WO 94/15562. Adhesives also can contain various additives, such as plasticizers, thickeners, and hydrocolloids.

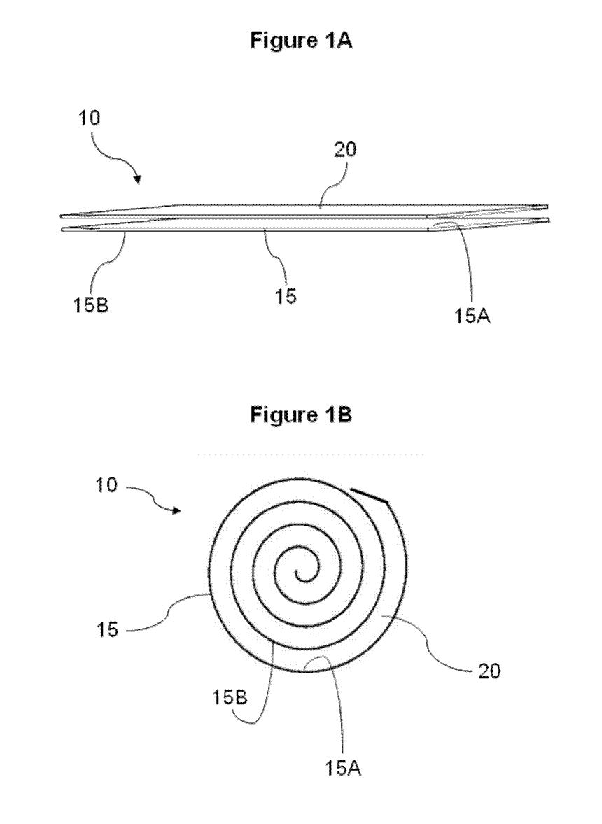

In some embodiments, a transfer adhesive or an adhesive provided on a double-coated tape (i.e., a tape having adhesive on both sides) can be used to attach a textile material to a subject. A transfer adhesive can be, for example, an adhesive coated onto one side of a liner that has two different release coatings. In some cases, the liner can be rolled up so that the adhesive is in contact with both surfaces of the liner and is essentially sandwiched between the two liner surfaces. As shown in FIGS. 1A and 1B, for example, transfer adhesive 10 can have release liner 15, with side 15A having release threshold A and side 15B having release threshold B, wherein release threshold A is higher than release threshold B. Adhesive 20 can be coated on side 15A of release liner 15, such that when transfer adhesive 10 is rolled up (FIG. 1B), adhesive 20 also comes into contact with side 15B of release liner 15. Unwinding the roll can cause adhesive 20 to release from side 15B of liner 15 (which has the lower release threshold), thus exposing one side of the adhesive. In the methods provided herein, a user (e.g., a health care worker or a subject to which the tape and textile material will be applied) can then attach adhesive 20 to a surface (e.g., a textile material), remove release liner 15 from the other side of adhesive 20, and apply the textile material to a second surface (e.g., the skin of the subject).

A double-sided tape (also referred to as double-coated tape) can be a film with adhesive (e.g., the same adhesive or two different adhesives) coated on both sides, and also can have a release liner on each side. When a double-sided tape is employed, a user can remove the liner from one side of the tape, attach the tape to a first surface (e.g., a textile material), remove the liner from the other side of the tape, and attach the material to a second surface (e.g., the skin of a subject).

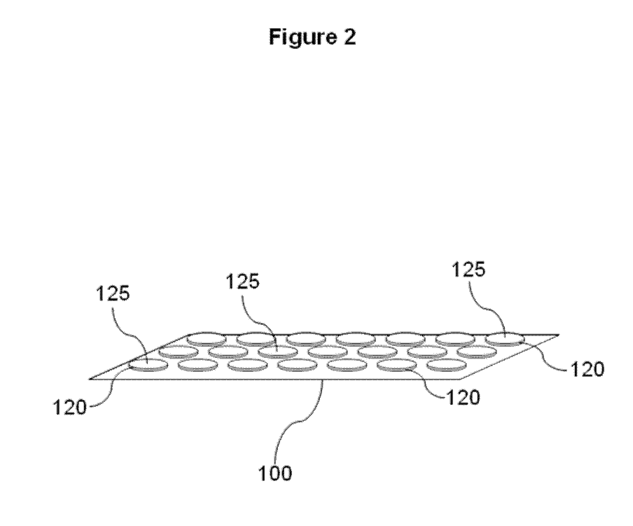

An adhesive tape or a transfer adhesive can be provided as a roll, a pad, or in any other suitable configuration. A roll of transfer adhesive or double-sided tape, for example, can have a width from 0.25 inch to 4 inches (or, 0.64 cm to 10.16 cm) (e.g., 0.25, 0.5, 0.75, 1, 1.25, 1.5, 2, 2.5, 3, or 4 inches) (or, e.g., 0.64, 1.27, 1.91, 2.54, 3.18, 3.81, 5.08, 6.35, 7.62, or 10.16 cm) and a length from 6 inches to 100 feet (or, 15.24 cm to 30.48 m) (e.g., 6, 12, 18, 20, 24, or 36 inches, or 4, 5, 10, 12, 16, 18, 20, 24, 30, 50, 75, or 100 feet) (or, e.g., 15.24, 30.48, 45.72, 50.88, 60.96, or 91.44 cm, or 1.22, 1.52, 3.05, 3.66, 4.88, 5.49, 6.10, 7.32, 9.14, 15.24, 22.86, or 30.48 m). A pad of transfer adhesive or double-sided tape can have any suitable shape, and can have a length, width, and/or diameter from 0.5 to 24 inches (or, 1.27 cm to 60.96 cm) (e.g., 0.5, 1, 2, 2.5, 3, 4, 6, 8, 10, 12, 14, 16, 18, 20, or 24 inches) (or, e.g., 1.27, 2.54, 5.08, 6.35, 7.62, 10.16, 15.24, 20.32, 25.40, 30.48, 35.56, 40.64, 45.72, 50.80, or 60.96 cm). Such rolls and pads can be cut into pieces of desired shapes and sizes before application of the adhesive or tape to textile material and skin. In some embodiments, pre-cut pieces of transfer adhesive or tape can be provided. As shown in FIG. 2, for example, a plurality of pre-cut pieces of adhesive 120 can be provided on release liner sheet 100. Pre-cut pieces 120 can be of uniform size and shape, or can be of varying sizes and shapes, such that a user can select the desired shape and size. Each pre-cut piece 120 can have a separate release liner 125 on the side opposite release liner 100, or the entire assembly of pieces 120 can be sandwiched between release liner sheet 100 and a second release liner sheet.

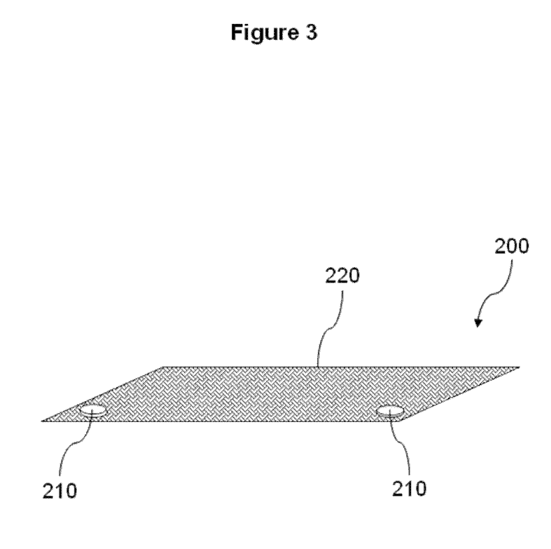

A transfer adhesive or double sided tape can be attached to one area of a textile material, or can be attached to more than one area (e.g., two, three, four, five, six, seven, eight, or more than eight areas) on the material. Typically, an adhesive or tape can be attached to one or more relatively small areas of the material, such that most of the surface area of the material can be in contact with the subject's skin after attachment. This can facilitate moisture absorption and keep the skin in contact with the antimicrobial component of the material, if an antimicrobial component is present. As depicted in FIG. 3, for example, textile material 200 can have two relatively small pieces of adhesive 210 attached thereto, with most of the surface area of textile material 200 not being in contact with the adhesive. Further, as shown in FIG. 3, adhesive 210 is attached to material 200 in an area that is not adjacent to edge 220, which can be positioned to protrude from a skin fold to allow for evaporation and translocation.

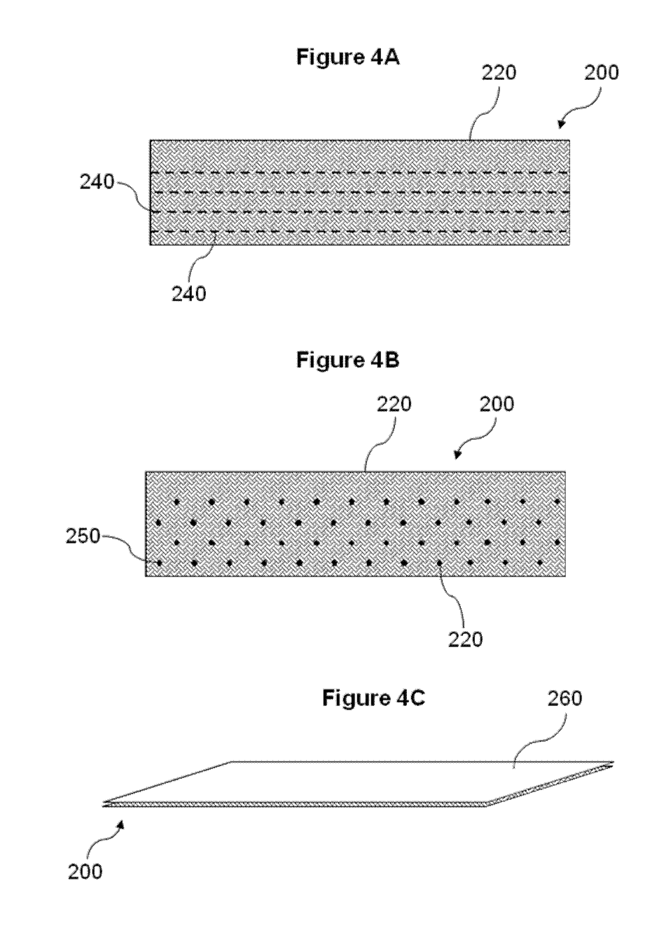

In some embodiments, a textile material can have an adhesive applied to one or both of its top and bottom surfaces during production. For example, an adhesive can be pattern coated onto a textile material, such that most (e.g., about 80 percent, about 85 percent, about 90 percent, about 95 percent, or about 98 percent) of the surface area remains uncoated by adhesive and available for direct contact with skin. The adhesive can be applied in any pattern (e.g., spots, stripes, or any other suitable pattern). As depicted in FIGS. 4A and 4B, for example, textile material 200 can have an adhesive coated thereon in dotted lines 240 (FIG. 4A) or dots 250 (FIG. 4B). In addition, as shown in FIGS. 4A and 4B, the adhesive may be applied such that it is not immediately adjacent to one or more edges (e.g., edge 220) of material 200, such that an adhesive-free area is available for positioning outside a skin fold. In embodiments where an adhesive is coated on a textile material during production, the material can have a release liner on the side(s) on which the adhesive is present. As depicted in FIG. 4C, for example, release liner 260 can be positioned on textile material 200 having an adhesive coating. Any suitable method can be used to coat an adhesive pattern on a textile material, including, without limitation, standard manufacturing methods known in the art (e.g., rotogravure, flexography, knife edge, etc.).

This document also provides articles of manufacture comprising one or more textile materials as described herein, as well as one or more means for attaching the material to the skin. For example, an article of manufacture can include a textile material and an adhesive tape (e.g., a roll or pad of transfer adhesive or double sided tape). In some embodiments, an article of manufacture also can include a scissors or other means for cutting the adhesive or tape into pieces of desired shapes and sizes. In some embodiments, an article of manufacture can include a textile material having an adhesive pattern coated onto one or both of its top and bottom surfaces. Such articles can include a scissors or other means for cutting the material into pieces of desired shapes and sizes. In any of the articles of manufacture provided herein, the textile material can include a polyurethane coating and/or an antimicrobial silver-containing compound, or can include neither a polyurethane coating nor an antimicrobial silver-containing compound.

The invention will be further described in the following example, which does not limit the scope of the invention described in the claims.

### Example

### Effectiveness of INTERDRY and Other Materials

INTERDRY3 Ag (Coloplast Corp.) is a polyurethane-coated polyester textile impregnated with an antimicrobial silver complex (0.06 mg silver sodium hydrogen zirconium phosphate per cm²). The material provides moisture transportation and translocation to keep skin dry, while the antimicrobial silver complex reduces odor. The material has demonstrated antimicrobial activity through a wear time of 5 days. In addition, the low-friction surface acts as a lubrication aid, thereby reducing skin-to-skin friction. As used in the experiments described herein, the material had dimensions of about 25.4 cm×365.8 cm, and contained about 21.9 mg of silver. The material was cut from a multi-use package designed to allow custom-fitting of skin folds.

The ability of the INTERDRY Ag (“INTERDRY”) material to absorb synthetic sweat, evaporate synthetic sweat, wick synthetic sweat under pressure, transport synthetic sweat, translocate synthetic sweat, slide between compressed sheets of synthetic skin, and withstand a pulling force was tested in comparison to BOUNTY® brand paper towels (“BOUNTY”), VIVA® brand paper towels (“VIVA”), MAIN STREET® brand paper towels (“MAIN STREET”), MEDLINE® HALOSHIELD® brand pillow covers (“MEDLINE”), and common, commercially-available pillow covers (“home pillow covers”).

TABLE 1
Brief description of tests used to determine
the abilities listed above.

| | | Procedural |
|---|---|---|
| Test | Function | Overview |
| Absorption | Absorb sweat | Fully submerged material, |
| | | drip dried for 10 minutes |
| Evaporation | Evaporate sweat | Material placed in 37° C. |
| | | oven for 3 hours, with |
| | | wicking application |
| Pressure | Absorb sweat | Material pressed between |
| Wicking | under pressure | synthetic skin |
| Translocation | Absorb sweat from one | Material submerged in one |
| | end of the material | beaker and hanging over |
| | and release it at the | the edge of another |
| | other end | beaker |
| Friction | Move between folds of | Use of Instron 5543 to |
| | skin with minimal | pull material through |
| | friction | compressed plates |
| Strength | Ability to withstand | Use of Instron 5543 to |
| | “wear and tear” | tear through 1 cm of |
| | | material |

### Testing Procedures

Synthetic sweat absorption: The material to be tested was cut to produce a piece that was one square inch (or, 6.45 square cm) in size. The piece was then placed into a weigh boat and the combined mass was measured on an analytical balance. After weighing, the material was submerged into a solution of synthetic sweat for 1 minute. The material then was hung by a paper clip to air dry. After 10 minutes, the material was placed back into the weigh boat and the combined mass was recorded. The difference between the initial combined mass and the final combined mass represented the amount of synthetic sweat absorbed.

Evaporation: The material to be tested was cut to produce a piece that was 4 cm×20 cm in size. Two 2-ounce jars were filled with 10 ml of synthetic sweat using a pipette and then capped with a screw top having a narrow opening for part of the material to penetrate. The mass of both jars and the material was measured on an analytical balance. After measurement, one end of the material was placed through the opening on one of jars so that it was in contact with the bottom of the jar, without slack. The other end of the material was placed through the opening of the other jar in the same manner so as to produce a bridge for the synthetic sweat to wick up both sides and stay saturated while evaporation occurred. The apparatus was placed inside a 37° C. oven. After 3 hours, the apparatus was removed from the oven and allowed to cool for an additional 15 minutes at room temperature. The final mass of the apparatus was then recorded. The difference between the final mass and initial mass represented the amount of synthetic sweat evaporated.

Pressure Wicking: The material to be tested was cut to produce a piece that was 16 cm×2 cm in size. The material was marked at the 15 cm point with ink from a pen. A medium VWR International weigh boat was filled to the top with synthetic sweat and placed at the edge of a steel plate that had been raised ˜1 inch off the counter top and was lying flat. A 16 cm×5 cm strip of synthetic skin was placed on top of the steel plate, followed by the material and another strip of synthetic skin. A piece of glass weighing 1.322 kg then was placed on top to further sandwich the material, with about 3½ cm of the material extending out from the sandwich. The end of the material was placed into the weigh boat, and the amount of time it took for the synthetic sweat to reach the 15 cm point was recorded. Two materials were tested simultaneously to balance the glass and distribute the weight. In addition, a trial was performed with an additional weight placed on top of the glass to give a total pressure increase relative to 4.0445 kg.

Translocation: A pipette was used to transfer 10 ml of synthetic sweat into a 50 ml beaker (A). The beaker (A) was placed on the edge of a ring stand so that it was approximately 2.5 cm above the counter top. A piece of material that was 1.2 cm wide and 12.5 cm in length was draped into the beaker (A) so that 1 cm of the material was in contact with the synthetic sweat and the other end was in a 50 ml or 15 ml beaker (B). The difference between the final and initial mass of beaker (B) was representative of the amount of synthetic sweat “translocated.”

Friction: The material to be tested was cut to produce a piece that is 16×3 cm in size. The material, as well as two sheets of synthetic skin that were 5×12 cm in size, were submerged in synthetic sweat. The synthetic skin was padded dry with a paper towel and both sides of the material were placed momentarily on a paper towel. The material was then sandwiched between the two pieces of synthetic skin. The material was further sandwiched by placing two stainless steel plates on both sides of the synthetic skin. A rubber band that was ˜1.5 mm thick and 19 cm in length (unstretched) was folded or twisted into two rubber bands and placed around the sandwich 2 cm from the top. A third stainless steel plate then was placed adjacent to the sandwich before it was locked into the stand at an Instron 5543 instrument. The Instron 5543 then recorded the average load required to completely remove the product from the stand.

Strength: The material to be tested was cut to produce a piece that was 6×8 cm in size. A hole was cut in the material so that the bolt for the Instron 5543 stand could fit through it, leaving 1 cm of the material to be ripped. The Instron 5543 then recorded the maximum load required to remove the material from the stand.

Results for each test procedure (below) are based on an average of three trials.

### Results

The test material absorbed more synthetic sweat than the MEDLINE and home pillow covers. It also absorbed more synthetic sweat than MAIN STREET, but it absorbed about 60% as compared to BOUNTY and VIVA. The ability of the material to evaporate synthetic sweat was about equal to that of the aforementioned paper towels, which was approximately 3 times greater than the MEDLINE and home pillow covers.

Under pressure, the textile material was shown to wick synthetic sweat at a greater rate than the other products tested. The material also showed an ability to absorb synthetic sweat at one end and release it at an opposite end. Of the known products tested, VIVA and MAIN STREET also showed this ability; specifically, MAIN STREET transferred a fractional amount while VIVA transported twice as much.

When moist, the textile material demonstrated an ability to slide between compressed synthetic skin with the least amount of friction. In addition, the textile material was found to have a tearing force that was twice as great as the MEDLINE and home pillow covers when subjected to a 1 cm tear length.

TABLE 2
Synthetic Sweat Absorption Data

| | | | Boat and | | Average |
|---|---|---|---|---|---|
| | | Boat and | Submerged | Sweat | Sweat |
| Product | Boat (g) | Wipe | Wipe | Absorbed | Absorbed |
| INTERDRY | 2.2171 | 2.3002 | 2.5693 | 0.2691 | 0.2803 |
| | 2.2178 | 2.3029 | 2.5925 | 0.2896 | |
| | 2.2746 | 2.3601 | 2.6423 | 0.2822 | |
| Home | 2.2615 | 2.3305 | 2.4473 | 0.1168 | 0.1279 |
| pillow | 2.2336 | 2.2987 | 2.4353 | 0.1366 | |
| cover | 2.2281 | 2.2962 | 2.4265 | 0.1303 | |
| VIVA | 2.2625 | 2.2967 | 2.8298 | 0.5331 | 0.5111 |
| | 2.2441 | 2.2936 | 2.805 | 0.5114 | |
| | 2.2121 | 2.2549 | 2.7438 | 0.4889 | |
| MAIN | 2.228 | 2.2543 | 2.4027 | 0.1484 | 0.2020 |
| STREET | 2.2574 | 2.2845 | 2.4929 | 0.2084 | |
| | 2.2262 | 2.2527 | 2.5019 | 0.2492 | |
| BOUNTY | 2.1958 | 2.2224 | 2.6537 | 0.4313 | 0.4462 |
| | 2.2388 | 2.2661 | 2.715 | 0.4489 | |
| | 2.2262 | 2.2546 | 2.713 | 0.4584 | |
| MEDLINE | 2.282 | 2.357 | 2.456 | 0.099 | 0.0870 |
| | 2.283 | 2.359 | 2.442 | 0.083 | |
| | 2.232 | 2.306 | 2.385 | 0.079 | |

TABLE 3
Evaporation Data

| | Initial | Final | Sweat Evap- | Average Sweat |
|---|---|---|---|---|
| Product | Mass (g) | Mass (g) | orated (g) | Evaporated (g) |
| INTERDRY | 196.4406 | 189.2322 | 7.2084 | 6.377 |
| | 197.3149 | 192.0994 | 5.2155 | |
| | 196.3071 | 189.5989 | 6.7082 | |
| Home | 195.2582 | 192.5773 | 2.6809 | 2.335 |
| pillow | 196.1017 | 193.7019 | 2.3998 | |
| cover | 195.7081 | 193.7838 | 1.9243 | |
| VIVA | 196.0705 | 189.5696 | 6.5009 | 6.353 |
| | 195.867 | 189.8407 | 6.0263 | |
| | 196.0726 | 189.5421 | 6.5305 | |
| MAIN | 196.2351 | 190.0328 | 6.2023 | 6.572 |
| STREET | 195.0011 | 187.7606 | 7.2405 | |
| | 196.6148 | 190.342 | 6.2728 | |
| BOUNTY | 194.9928 | 189.3779 | 5.6149 | 6.235 |
| | 195.9368 | 188.4445 | 7.4923 | |
| | 195.6535 | 190.0543 | 5.5992 | |
| MEDLINE | 195.8845 | 193.8139 | 2.0706 | 2.375 |
| | 196.0428 | 193.381 | 2.6618 | |
| | 195.8181 | 193.4255 | 2.3926 | |

TABLE 4
Pressure Wicking Data.

| | Weight | Time |
|---|---|---|
| Product | (g) | (sec) |
| INTERDRY | 0 | 168 |
| | 1322 | 192 |
| | 4044 | 201 |
| VIVA | 0 | 335 |
| | 1322 | 346 |
| | 4044 | 435 |
| MAIN | 0 | 520 |
| STREET | 1322 | 875 |
| | 4044 | 1050 |
| BOUNTY | 0 | 600 |
| | 1322 | 373 |
| | 4044 | 482 |
| MEDLINE | 0 | 3000 |
| | 1322 | 1115 |
| | 4044 | 1597 |

TABLE 5
Translocation Data

| | Initial | Final | Sweat | Average |
|---|---|---|---|---|
| | Beaker | Beaker | Trans- | Sweat Trans- |
| Product | Mass (g) | Mass (g) | ported (g) | ported (g) |
| INTERDRY | 13.9906 | 15.229 | 1.2384 | 1.2405 |
| | 14.0327 | 15.0429 | 1.0102 | |
| | 13.5958 | 15.0687 | 1.4729 | |
| Home | 31.8471 | 31.8471 | 0 | 0 |
| pillow | 9.2796 | 9.2796 | 0 | |
| cover | 30.8348 | 30.8348 | 0 | |
| VIVA | 9.0787 | 10.6272 | 1.5485 | 2.4619 |
| | 9.0956 | 12.0689 | 2.9733 | |
| | 13.7556 | 15.7061 | 1.9505 | |
| MAIN | 14.1556 | 14.4499 | 0.2943 | 0.3193 |
| STREET | 13.5336 | 13.8578 | 0.3242 | |
| | 14.3691 | 14.6835 | 0.3144 | |
| BOUNTY | 13.9787 | 13.9787 | 0 | 0 |
| | 14.0016 | 14.0018 | 0 | |
| | 13.6854 | 13.6854 | 0 | |
| MEDLINE | 29.194 | 29.194 | 0 | 0 |

TABLE 6
Friction Data

| | | | Average- | Average |
|---|---|---|---|---|
| | Average | Maximum | Average | Maximum |
| Product | Load (N) | Load (N) | Load (N) | Load (N) |
| INTERDRY | 1.43 | 2.64 | 1.41 | 2.67 |
| | 1.4 | 2.63 | | |
| | 1.4 | 2.74 | | |
| Home | 2.01 | 2.6 | 2.08 | 2.61 |
| pillow | 2.02 | 2.49 | | |
| cover | 2.22 | 2.74 | | |
| VIVA | break | 2.63 | — | 2.66 |
| | break | 2.68 | — | |
| | break | 2.68 | — | |
| MAIN | break | 2.26 | — | 1.59 |
| STREET | break | 1.24 | — | |
| | break | 1.26 | — | |
| BOUNTY | break | 2.42 | — | 2.22 |
| | break | 2.39 | — | |
| | break | 1.85 | — | |
| MEDLINE | 2.44 | 3.25 | 2.29 | 3.13 |
| | 2.2 | 3.17 | | |
| | 2.22 | 2.98 | | |

TABLE 7
Strength Data

| | | Average |
|---|---|---|
| | Maximum | Maximum |
| Product | Load (N) | Load (N) |
| INTERDRY | 63.66 | 67.26 |
| | 74.83 | |
| | 63.3 | |
| Home | 39.89 | 32.95 |
| pillow | 30.89 | |
| cover | 28.06 | |
| VIVA | 2.14 | 1.61 |
| | 1.23 | |
| | 1.46 | |
| MAIN | 1.45 | 1.19 |
| STREET | 0.76 | |
| | 1.36 | |
| BOUNTY | 2.55 | 2.33 |
| | 1.86 | |
| | 2.58 | |
| MEDLINE | 39.86 | 34.20 |
| | 28.22 | |
| | 34.51 | |

TABLE 8
Data Analysis/Comparison.

| | | | Synthetic sweat | | |
|---|---|---|---|---|---|
| | Synthetic sweat | | evaporated at 37° C. | | |
| | absorbed per | Synthetic sweat | through wicking | Tearing | Friction |
| Product | square inch (g) | transported (g) | ability of material (g) | force (N) | force (N) |
| INTERDRY | 0.28 | 1.2 | 6.4 | 67.3 | 1.4 |
| Home pillow | 0.13 | 0 | 2.3 | 33.0 | 2.1 |
| cover | | | | | |
| MEDLINE | 0.09 | 0 | 2.4 | 34.2 | 3.1 |
| BOUNTY | 0.45 | 0 | 6.2 | 2.3 | 2.2 |
| MAIN | 0.20 | 0.3 | 6.6 | 1.2 | 1.6 |
| STREET | | | | | |
| VIVA | 0.51 | 2.5 | 6.4 | 1.6 | 2.7 |
| (control | — | — | 0.4 | — | — |
| blank) | | | | | |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A textile material having top and bottom surfaces and comprising a polyurethane coating, wherein said textile material is adapted for placement within a skin fold for management of one or more skin health complications associated therewith, and wherein said textile material has an adhesive on at least one of the top and bottom surfaces.

2. (canceled)

3. The textile material of claim 1, wherein the textile material is impregnated with an antimicrobial silver complex.

4. The textile material of claim 3, wherein said antimicrobial silver complex comprises one of a silver ion-exchange resin, a silver zeolite, a silver exchanged on calcium phosphate, and a silver glass.

5. The textile material of claim 3, wherein said antimicrobial silver complex is silver sodium hydrogen zirconium phosphate.

6. The textile material of claim 3, wherein said antimicrobial silver complex is present at a concentration between 0.01 to 0.10 mg/cm2.

7. (canceled)

8. The textile material of claim 1, wherein said adhesive is pattern coated onto only one of the top and bottom surfaces of said textile material.

9. The textile material of claim 1, wherein said adhesive is selected from a group of adhesives consisting of silicone adhesives; acrylic adhesives; hydrogel adhesives; and hydrocolloid adhesives.

10. The textile material of claim 1, wherein at least a portion of a composition of said adhesive is selected from a group consisting of: caoutchouc and caoutchouc-like synthetic homo-, co-, and block-copolymers; polyacrylate and copolymerisates thereof; polyurethane; silicone; polyphenylene oxide; acrylate blockcopolymer polyisobutylene; and polyvinyl ether.

11. The textile material of claim 10, wherein said composition of said adhesive includes zinc oxide.

12. (canceled)

13-14. (canceled)

15. The textile material of claim 1, wherein said adhesive comprises an adhesive coated onto one side of a liner that has two different release coatings.

16. (canceled)

17. A method for using a textile material for management of one or more skin health complications associated with a skin fold, comprising the steps of:
placing a piece of adhesive onto a textile material dimensioned such that at least one portion of said textile material is exposed to air outside of the skin fold; and
placing said textile material over skin of the skin fold, such that said adhesive is in contact with the skin, and such that the skin fold envelopes said textile material with said at least one portion thereof being exposed to air.

18. The method of claim 17, wherein said textile material comprises a polyester textile coated with polyurethane and impregnated with an antimicrobial silver complex.

19. The method of claim 18, wherein said antimicrobial silver complex is one of a silver ion-exchange resin, a silver zeolite, a silver exchanged on calcium phosphate, and a silver glass.

20. The method of claim 18, wherein said antimicrobial silver complex is silver sodium hydrogen zirconium phosphate.

21. (canceled)

22. The method of claim 17, further comprising:
cleansing the skin fold with a skin cleanser that increases a translocation function of said textile material.

23-26. (canceled)

27. An article of manufacture comprising:
a textile material having top and bottom surfaces and comprising a polyurethane coating, wherein said textile material is adapted for placement within a skin fold for management of one or more skin health complications associated therewith; and
an adhesive attaching the textile material to a pad.

28-34. (canceled)

35. An article of manufacture comprising:
a textile material having top and bottom surfaces, said textile material comprising a polyurethane coating and an antimicrobial silver complex, wherein said textile material is adapted for placement within a skin fold for management of one or more skin health complications associated therewith, and wherein said antimicrobial silver complex is silver sodium hydrogen zirconium phosphate; and
an adhesive applied to the textile material and configured to allow the textile material to be rolled into a roll.

36-39. (canceled)
